(19) United States
(12) Patent Application Publication
(10) Pub. No.: US 2021/0307593 A1
(43) Pub. Date: Oct. 7, 2021

(54) Protective Sheath for Bronchoscope

(71) Applicant: J & J Medical, LLC, Ferriday, LA (US)

(72) Inventors: Joshua A. Foster, Ferriday, LA (US); Jerred Anderson, Winnsboro, LA (US)

(73) Assignee: J & J Medical, LLC, Ferriday, LA (US)

(21) Appl. No.: 17/125,194

(22) Filed: Dec. 17, 2020

Related U.S. Application Data
(60) Provisional application No. 63/005,555, filed on Apr. 6, 2020.

(51) Int. Cl. A61B 1/00; A61B 1/267; A61B 1/04; A61B 1/005

(52) U.S. Cl. CPC A61B 1/00135; A61B 1/2676; A61B 1/0051; A61B 1/0014; A61B 1/04; A61B 1/00094

(57) ABSTRACT

A protective sheath for a bronchoscope is provided, comprising a flexible and collapsible sleeve having a first open end and a second open end; wherein the first open end includes a first resilient member adapted to sealably connect to a bronchoscope control unit; and wherein the second open end includes a second resilient member adapted to sealably connect to an adapter device for receiving the bronchoscope. The sleeve is constructed from a nonpermeable, collapsible, and food grade plastic. A suction port is preferably attached to the sleeve adapted to fluidically remove air and fluids from the sleeve during the bronchoscopy procedure.

### CLAIM OF BENEFIT TO PRIORITY APPLICATION

This nonprovisional application claims the benefit under 35 USC § 119(e) to U.S. provisional application, Ser. No. 63/005,555, filed on Apr. 6, 2020.

### BACKGROUND OF THE INVENTION

### 1. Field of the Invention

The present invention relates generally to protective sheaths which prevent body fluids from a patient's lungs from contacting a healthcare provider during a bronchoscopy procedure, and more particularly to collapsible and disposable protective sheaths which include a suction port to enable suctioning of the air and body fluids from inside the sheath during the procedure.

### 2. Description of Related Art

Bronchoscopy allows for visualization of the lower airway structures for either diagnostic or therapeutic procedures. The flexible bronchoscope slides in and out of a rubberized connector attached to the proximal portion of an endotracheal tube. Bronchoscopy includes an evaluation of the respiratory system, including larynx, trachea, and the bronchi for evidence of abnormalities. The physician can see these structures during different stages of the breathing cycle, and the images and video from the procedure can be recorded for assessing the patient's condition. Bronchoscopy is usually performed by a pulmonologists, surgeon, or intensivist. Although a bronchoscope does not allow for direct viewing and inspection of the lung tissue itself, samples of the lung tissue can be biopsied through the bronchoscope for examination in the laboratory.

The most common form of bronchoscope is a flexible fiber optic bronchoscope. This instrument includes a proximal handle with camera control features and an elongated flexible portion that terminates at its distal end in a fiber optic camera and light. The flexible portion may also include channels for air/water or for biopsy/suction functions. During the bronchoscopy, the user can see the tissues of the airways either directly by looking through the instrument or by viewing on a monitor. When a patient is intubated, i.e. an endotracheal tube is already in place for mechanical ventilation. An adapter is connected to the end of the endotracheal tube, and the adapter further includes a mechanical ventilation port and an insertion port for entry of the bronchoscope. The insertion port typically includes a rubberized opening that conforms to the outside of the bronchoscope to serve as a slidable seal while the bronchoscope is moved into and out of the adapter. The flexible fiber optic bronchoscope is passed through the insertion port on the adapter and through the endotracheal tube. Once the bronchoscope exits the endotracheal tube, it is further guided and oriented to navigate into individual lobar or segmental bronchi to view the patient's lung conditions.

During the bronchoscopy, positive pressure from the mechanical ventilator results in body fluids and secretions from the patient's lungs being forced beyond the slidable seal of the insertion port, i.e. the liquid and aerosolized body fluids are covering the bronchoscope itself and are being released to the atmosphere around the physician, nurses, and other persons involved in the procedure. Those body fluids may contain bacterial or viral contaminants which are highly contagious to others, and they present a significant risk for infection for healthcare providers. There are approximately 500,000 bronchoscopies performed yearly in the United States, exposing exponential numbers of healthcare providers to droplet and aerosolization of potentially harmful pathogens. These pathogens include tuberculosis, pseudomonas, and our current threat COVID 19.

In view of such risks, it is desired to have a protective device that effectively prevents healthcare providers from becoming exposed to those body fluids which exit the adapter. We have developed a device that limits the healthcare provider's exposure considerably. The present invention is a device that encompasses the flexible bronchoscope from the rigid hand piece of the scope to the endotracheal tube with a side suction port. During the procedure as the scope is moved in and out of the endotracheal tube connector, the sleeve catches and encapsulates droplets sprayed from the airway. At the end of the procedure, components of the endotracheal tube connector are closed with the sleeve in place, keeping further expulsion of droplets from the airway from coming out. The sleeve can then be removed with the suction system in place drawing negative pressure on the sleeve and preventing enclosed contaminants from going into the environment.

Therefore, a protective sheath for bronchoscopes is provided that includes a first distal end that sealably connects to the camera/control unit and a second distal end that sealably connects to the adapter. When installed, the flexible portion of the bronchoscope is fully surrounded by the protective sheath, and any body fluids which escape from the adapter are safely contained within the sheath. The sheath is collapsible to accommodate the changing distance between the adapter and the control unit as the flexible portion is inserted into and withdrawn from the lungs. The sheath would have the further benefit of protecting against external contaminants contacting the bronchoscope tube and entering the patient.

### SUMMARY OF THE INVENTION

In summary, the present invention is a protective sheath for a bronchoscope, comprising a flexible and collapsible sleeve having a first open end and a second open end; wherein the first open end includes a first resilient member adapted to sealably connect to a bronchoscope control unit; and wherein the second open end includes a second resilient member adapted to sealably connect to an adapter device for the bronchoscope.

Preferably, the sleeve is constructed from a nonpermeable, collapsible, and food grade plastic sufficient to contain air and fluids.

More preferably, the first and second resilient members each comprise a circular member having an outer circumferential groove, and wherein the first and second open ends of the sleeve are sealably retained within the outer circumferential groove of their respective circular members by a resilient O-ring.

More preferably, one or more of the circular members includes a surrounding and releasable clamp adapted to further secure the protective sheath between the bronchoscope control unit and the bronchoscope adapter.

The protective sheath may further include a suction port operatively attached to the sleeve adapted to fluidically remove air and fluids from the sleeve.

A bronchoscope kit is also provided, comprising a bronchoscope having a control unit and a flexible camera portion; a bronchoscope adapter having a bronchoscope insertion port; a protective sheath for a bronchoscope, comprising a flexible and collapsible sleeve having a first open end and a second open end; wherein the first open end includes a first resilient member adapted to sealably connect to the bronchoscope control unit; and wherein the second open end includes a second resilient member adapted to sealably connected to the bronchoscope insertion port of the adapter.

These and other advantages of the present invention are explained in more detail in the following specification and drawings.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements.

FIG. 1 illustrates a view of one embodiment of the present invention as a protective sheath for a bronchoscope.

FIG. 2 illustrates the embodiment of FIG. 1 shown in an operable configuration surrounding the flexible portion of a bronchoscope and sealably connected between the camera control unit and the adapter.

FIG. 3 illustrates a detailed view of the circular resilient member and optional releasable clamp for securing the ends of the sheath to either the bronchoscope control unit and/or the bronchoscope adapter.

FIG. 4 illustrates a detailed end view of the resilient member surrounded by the releasable clamp.

FIG. 5 illustrates a detailed view of the resilient member with O-ring used to secure the plastic sleeve to the resilient member.

### DETAILED DESCRIPTION OF THE INVENTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed. In addition, a specific scale of operation is described as the current illustrative embodiment; however, the scale can be increased or decreased by simply adjusting the size of the various parts of the apparatus as will be apparent to one of skill in the art, and no limitation of the scale of operation is intended by this specification.

In describing and claiming the present disclosure, the following terminology will be used in accordance with the definitions set out below. As used in this specification and the appended claims, the singular forms “a,” “an,” and “the” include plural referents unless the context clearly dictates otherwise. As used herein, the terms “comprising,” “including,” “having,” “containing,” “characterized by,” and grammatical equivalents thereof are inclusive or open-ended terms that do not exclude additional, unrecited elements or method steps.

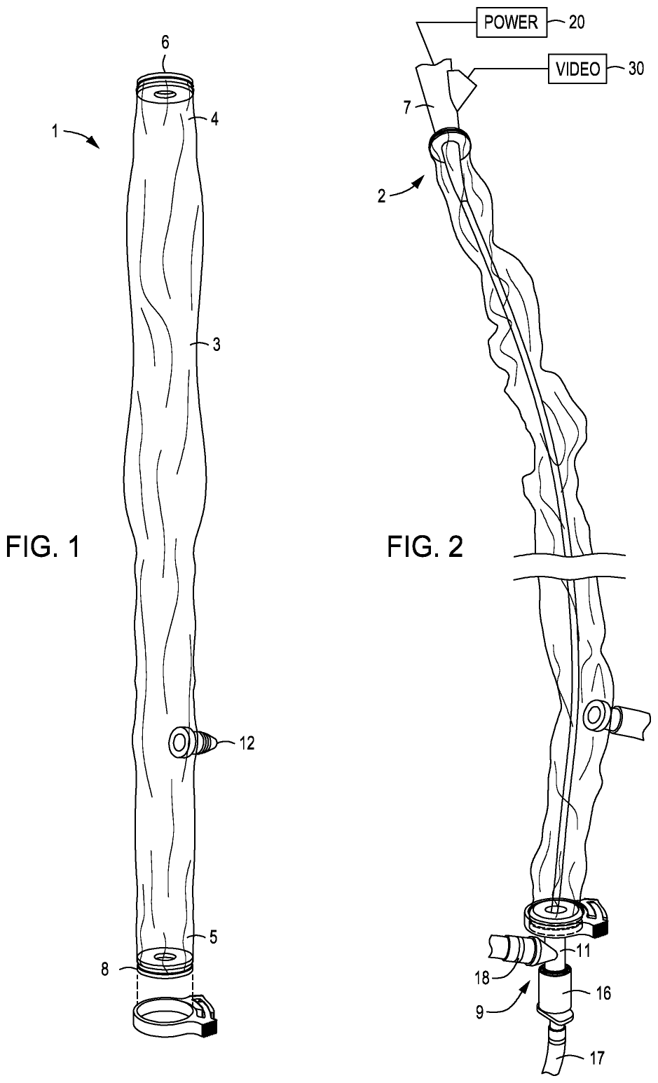

With reference to a preferred embodiment of the invention shown in FIGS. 1 and 2, a protective sheath 1 for a bronchoscope 2 is shown to generally comprise a flexible, collapsible plastic sleeve 3 having a first open end 4 and a second open end 5. The first open end 4 includes a first circular resilient member 6 adapted to sealably connect to a bronchoscope control unit 7. The bronchoscope control unit 7 is conventionally connected to a power source 20 and may also be connected to a video monitor 30. Similarly, the second open end 5 includes a second circular resilient member 8 adapted to sealably connected to an adapter device 9 for the bronchoscope 2. The adapter 9 generally includes a bronchoscope insertion port 11, an endotracheal (ET) tube port 16 having an ET tube 17 installed into the patient, and a mechanical ventilation port 18 connected to a ventilator and other anesthesia equipment.

When installed, the protective sheath 1 surrounds the flexible portion 15 of the bronchoscope 2 to sealably contain all air and fluids which may exit the adapter 9 as a result of positive pressure from the lungs and the ventilator, while also preventing contaminants outside the sheath 1 from entering the adapter 9 or the lungs of the patient.

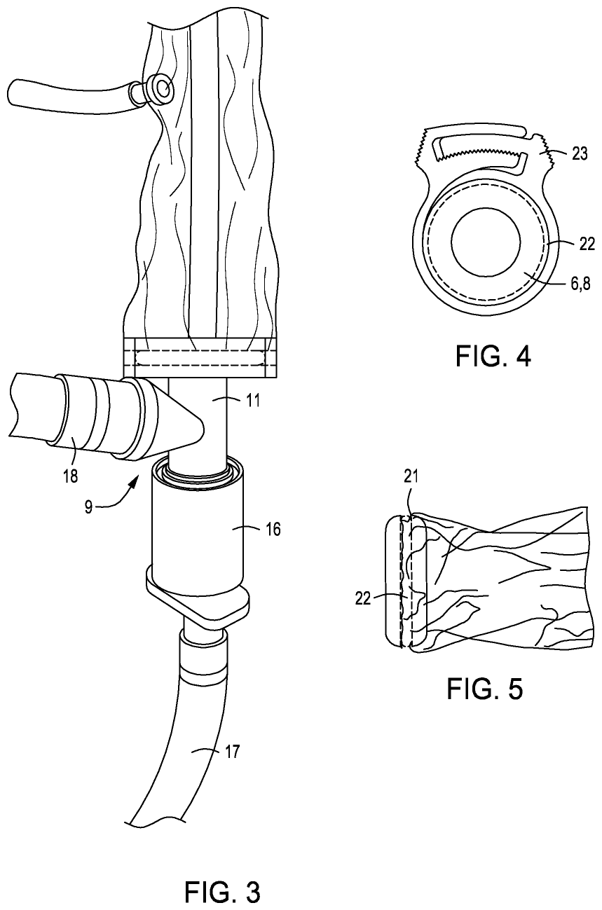

The sealable feature of the resilient members 6, 8 can be accomplished by a wide variety of structures. For example, the resilient members 6, 8 can be constructed of a material that stretches and conforms to the bronchoscope insertion port 11 on the adapter 9 or a similar structure on the control unit 7, such as shown in FIG. 2. Alternatively, the members 6, 8 may be threaded clamps which threadably mate with corresponding portions of the adapter 9 and the control unit 7, respectively. In a more preferred embodiment shown in more detail in FIGS. 4 and 5, the resilient members 6, 8 each comprise a circular member having an outer circumferential groove 21, and wherein the first and second open ends 4, 5 of the sleeve 3 are sealably retained within the outer circumferential groove 21 of their respective circular members by a resilient O-ring 22. Furthermore, one or more of the resilient members 6, 8 may include a surrounding and releasable clamp 23, such as a “snap grip” hose clamp as shown in FIGS. 3 and 4, adapted to further secure the protective sheath 1 between the bronchoscope control unit 7 and the bronchoscope adapter 9.

The sleeve 3 is preferably constructed from a nonpermeable, collapsible, and food grade plastic that is sufficient to contain liquids and aerosolized body fluids and secretions within the protective sheath 1 while it is securely connected around the bronchoscope 2 between the control unit 7 and the adapter 9.

A suction port 12 is optionally and sealably attached to the sleeve 3 and adapted to fluidically remove air and fluids from the sleeve 3 during the bronchoscopy procedure using conventional suction equipment available in the surgical environment.

In operation, the first resilient member 6 of the protective sheath 1 is guided over the flexible portion 15 of the bronchoscope 2 and then securely connected to the control unit 7. Then, the second resilient member 8 is securely connected to the bronchoscope insertion port 11 of the adapter 9. Alternatively, the second resilient member 8 can first be connected to the bronchoscope insertion port 11, and then the first resilient member 6 can be guided over the flexible portion 15 of the bronchoscope 2 and then secured to the control unit 7. The suction port 12 is then connected to the suction equipment available in the operating room. After completing either method of installation, the flexible portion 15 of the bronchoscope 2 can then be inserted into the bronchoscope insertion port 11 and the bronchoscopy procedure can be conducted normally. During the procedure, the attached suction port 12 can be employed to remove air and fluids from the sleeve 3, which advantageously collapses the sleeve 3 around the flexible portion 15 of the bronchoscope 2 so that the sleeve 3 does not interfere with manipulation and control of the bronchoscope 2 during the procedure

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only, and the scope of the present invention is to be limited only by the following claims.

The invention claimed is:

1. A protective sheath for a bronchoscope, comprising:
a flexible and collapsible sleeve having a first open end and a second open end;
wherein the first open end includes a first resilient member adapted to sealably connect to a bronchoscope control unit; and
wherein the second open end includes a second resilient member adapted to sealably connected to an adapter device for the bronchoscope.

2. The device of claim 1, wherein the sleeve is constructed from a nonpermeable plastic sufficient to contain air and fluids.

3. The device of claim 1, wherein the first and second resilient members each comprise a circular member having an outer circumferential groove, and wherein the first and second open ends of the sleeve are sealably retained within the outer circumferential groove of their respective circular members by a resilient O-ring.

4. The device of claim 3, wherein one or more of the circular members includes a surrounding and releasable clamp adapted to further secure the protective sheath between the bronchoscope control unit and the bronchoscope adapter.

5. The device of claim 1, further including a suction port operatively attached to the sleeve adapted to fluidically remove air and fluids from the sleeve.

6. A bronchoscope kit, comprising:
a bronchoscope having a control unit and a flexible camera portion;
a bronchoscope adapter having a bronchoscope insertion port;
a protective sheath for a bronchoscope, comprising a flexible and collapsible sleeve having a first open end and a second open end; wherein the first open end includes a first resilient member adapted to sealably connect to the bronchoscope control unit; and wherein the second open end includes a second resilient member adapted to sealably connect to the bronchoscope insertion port of the adapter.

7. The kit of claim 6, wherein the sleeve is constructed from a nonpermeable plastic sufficient to contain air and fluids.

8. The kit of claim 6, wherein the first and second resilient members each comprise a circular member having an outer circumferential groove, and wherein the first and second open ends of the sleeve are sealably retained within the outer circumferential groove of their respective circular members by a resilient O-ring.

9. The kit of claim 8, wherein one or more of the circular members includes a surrounding and releasable clamp adapted to further secure the protective sheath between the bronchoscope control unit and the bronchoscope adapter.

10. The kit of claim 6, further including a suction port operatively attached to the sleeve adapted to fluidically remove air and fluids from the sleeve.
